(19) United States
(12) Patent Application Publication
(10) Pub. No.: US 2016/0287151 A1
(43) Pub. Date: Oct. 6, 2016

(54) DEVICE AND METHOD AND FOR THE IN-VIVO, NON-INVASIVE MEASUREMENT OF THE OSMOLALITY OF BIOLOGICAL TISSUE FLUID, UTILIZING REFLECTION OF MULTI-FREQUENCY ELECTROMAGNETIC WAVES IN THE RADIO-FREQUENCY RANGE

(71) Applicant: Menachem Margaliot, Bnei Brak (IL)

(72) Inventor: Menachem Margaliot, Bnei Brak (IL)

(21) Appl. No.: 14/676,908

(22) Filed: Apr. 2, 2015

(51) Int. Cl. A61B 5/145; A61B 5/00

(52) U.S. Cl. CPC A61B 5/14546; A61B 5/4875; A61B 5/742; A61B 5/681

(57) ABSTRACT

A system (2) for non-invasive measurement of the osmolality of biological tissue fluid in a living specimen (3) includes an RF generator for generating RF signals and transmitting these via an appropriate antenna towards tissue of a living specimen. An RF receiver (8) is coupled to another antenna, for receiving and measuring the intensity the RF signals reflected from aforementioned tissue, and for feeding said intensity to a processor (10) for comparison of relative reflection intensities of at least two RF frequencies impinging on the tissue with a reference reflection ratio signal.

### CROSS-REFERENCE TO OTHER APPLICATIONS

This application claims priority from U.S. Provisional Patent Application 61/977647, filed Apr. 10, 2014, the contents of which are incorporated herein by reference.

### FIELD OF THE INVENTION

The present invention relates generally to a system and a method for non-invasively measuring the osmolality of the body fluids in a living specimen. The term “living specimen” as used herein is intended to define living tissue of a human, an animal or a plant.

### BACKGROUND OF THE INVENTION

Osmolality of biological systems is the concentration of solutes in body water. Many biological processes in living systems consist of passage of living cells membranes by various molecules, due to osmotic pressure. Hence, the correct osmotic pressure is a critical parameter in the proper function of the physiological system.

Water is the solvent in all known living systems, and thus, the correct hydration level of the living system is essential for maintaining the correct osmolality. The human body gains water by drinking and extracts water from solid food. It loses water mainly by sweating and urinating. Under normal conditions, the output and input of water are balanced by thirst, which prompts drinking, but this is insufficient in many cases, especially under conditions of heat stress, strong physical effort and even more so—under the combination of the two.

Water deficiency (dehydration) results in an increase in the body fluids osmolality, which, in turn, causes malfunctioning of many body functions, and when excessive can be fatal.

On the other hand, over hydration is uncommon in healthy human beings, but is a common result of renal insufficiency or other cardiovascular diseases. It also may occur during dialysis treatment of kidney diseases and may have serious consequences.

The measurement of the body fluid osmolality (namely: the hydration state) of living specimen is thus of great value for the well-being of said living specimen.

Bio-impedance has been suggested in the prior art to measure non-invasively human hydration level. Examples include the following:
1. Lozano-Nieto A.: Impedance ratio in bioelectrical impedance measurements for body fluid shift determination,—Proceedings of the IEEE annual north-east bio engineering conference, Apr. 9-10, 1998
2. U.S. Pat. No. 4,364,008, Jacques 1982
3. U.S. Pat. No. 4,488,559, Iskander 1984
4. U.S. Pat. No. 5,767,685 Walker 1998

The systems thus described consist of measurement of the electric impedance between a pair (or pairs) of electrodes, attached to the skin of the examined person at distant locations (a common approach is to attach one electrode to the wrist of the examined person, while the other electrode is attached to the opposite ankle).

Although the bio-impedance based devices provide osmolality or hydration state information, they suffer severe limitations. For example, the strongest contributor to the impedance thus measured is the upper layer of the skin. The measurement results are thus extremely dependent on the skin condition, which is dependent in turn on various factors, such as skin thickness, external temperature, the size of the examined person and his/her body composition (fat versus muscle versus bone mass etc.) which do not depend on the hydration state. This limits the bio-impedance methods to be applicable only as a shift detector in hydration state, namely: the shifts in the hydration condition of the examined person from the starting point on, provided the external conditions (temperature, humidity) remain constant.

Absolute hydration levels cannot be thus measured. In some cases bio-impedance measurements can be dangerously misleading. An example of such a situation is an attempt to measure the hydration state of a person during heavy physical effort: the skin might be still rather hydrated due to strong sweating, indicating a heavily over-hydrated state, while in fact the person might be badly dehydrated.

In contrast, the present invention utilizes non-contact penetration and sub-sequent reflection of radio-waves from a depth of up to about 1 cm. The upper layer of the skin (due to its very high impedance) is rather transparent to radio wave has therefore a very limited effect on the reflection process.

Radio wave absorption:

A more advanced approach is presented in U.S. Pat. No. 6,849,046—Eyal-Bickels et. al., 2005, which describes using radio waves penetrating the examined tissue, and measures the intensity of the waves after passing a given organ, namely, the attenuation of the waves by the examined tissue.

This method is not critically dependent on skin condition, but very dependent on the size of the examined organ and its exact composition (muscle, fat and bone).

In contrast to that, the present invention depends on reflection from a depth of about 1 cm only and provides information which on one hand indicates the osmolality in tissue below the upper skin but on the other hand does not dependent on organ size and composition, especially when applied to muscular regions in the limbs.

### SUMMARY OF THE INVENTION

The present invention seeks to provide an improved electromechanical hasp lock assembly, as described more in detail hereinbelow.

It is therefore a broad object of the present invention to provide a system and method for non-invasively measuring the osmolality, and hence—the hydration state in a living specimen, and to provide a warning signal upon the detection of a deviation from the normal state.

Said system comprising an RF transmitter for emitting RF radiation signals at a plurality of frequencies, and for transmitting said radiation signals towards a tissue of a living specimen; an RF receiver to receive the RF signals reflected from said tissue; a microprocessor, to which the intensities of both the transmitted and reflected said RF signals are fed, said microprocessor computing from the ratios of the intensities of said reflected signals at the various frequencies, the osmolality in said tissue; an indicator unit for providing an output signal representative of the osmolality or hydration level in said tissue and providing an alarm in case the result thus obtained is out of the normal range for this kind of tissue.

It is well known to those skilled in the interactions between electromagnetic waves in the radio-frequency range (RF waves) and living tissue, that RF waves impinging on tissue are partially reflected. The reflection mechanism is based on the fact that RF waves impinging on and penetrating tissue induce cyclic motion of the electrical charges in the tissue. The well-known result provided by Maxwell's electromagnetic theory is emission of RF waves from said charges at the same frequency as the impinging waves, but in different directions. This process of reflection is the basis for many technologies such as the human detection by radars.

The exact mechanism of reflection is however frequency dependent:
at frequencies below approximately 2 GHz, (“low” frequencies in the following) the impinging RF waves induces mainly ionic forth and back motion of the ions dissolved in the body water, and hence—the RF reflection is produced by said linear motion of these ions. The intensity of the reflected waves has a positive correlation to the quantity of ions dissolved in the tissue member under examination. At higher RF frequencies (above ˜1.5 GHz and up to 300 GHz—“high frequencies” in the following), the motion induced in the dissolved ions becomes weak and hence—the intensity of the reflected waves produced by this process is also weakened. However, at this frequencies range, another process takes place: water molecules possess an electric dipole, and in response to the cyclic RF wave impinging, the water molecules rotate (to align the dipole direction with the cyclic electric field of the impinging waves).

This rotation of the water molecule also produces a reflected RF wave, the intensity of which is positively correlated to quantity of water molecules in the tissue member under examination. Comparing the intensities of the reflected waves at the “low” frequencies to those at the “high” frequencies enables the computation of the quantity of water versus quantity of dissolved ions in the tissue member under examination, namely, the osmolality or the hydration level in the fluids of this tissue member.

A system for non-invasive in-vivo, measurement of the osmolality of biological tissue fluid in a living specimen, the system including:
an RF generator/transmitter for emitting RF radiation signals at a plurality of frequencies for transmitting the radiation signals towards a tissue of a living specimen, via an appropriate antenna,
a receiver adapted to receive RF radiation Signals reflected from the tissue of living specimen at the plurality of frequencies via an appropriate antenna, and
a processor unit, which initiates operation of the transmitter, receives signals from the receiver and compares the intensity of the RF signals at the plurality of frequencies reflected from the tissue, with that of at least one reference value obtained from tissue with a known normal osmolality, and based on that, computes the osmolality of the tissue under examination. This information is transferred to an indicator unit for providing an output signal representative of the osmolality of the fluid of the tissue, and issuing a warning signal if the osmolality value is significantly out of normal range.

In accordance with non-limiting embodiments of the invention:
The generator/transmitter emits RF radiation at frequencies of between 10 kHz and 300 GHz.

The receiver is adapted to receive a signal reflected from the tissue.

The indicator unit provides an audible, visual or palpating output signal. The indicator might be a standalone device such as an electronic wrist watch, in which the hydration measurement device is incorporated, and the watch's display presents the wearers osmolality, or alternatively—the raw data thus obtained in the wrist watch is transmitted (by a Bluetooth or similar communication tool) to a cell-phone which serves as the presentation tool.

There may be a suitable attachment for attaching the system to a part of the user's body.

The plurality of frequencies include at least one frequency at which RF energy impinging on tissue induces mainly ionic motion of ions dissolved in the tissue water.

The plurality of frequencies include a frequency around 40 MHz.

The plurality of frequencies include at least one frequency at which RF energy causes mainly the rotational excitation of tissue water molecules.

The plurality of frequencies include a frequency around 2.4 GHz.

The plurality of frequencies includes more than two frequencies.

The indicator unit provides a warning signal when the osmolality of the living specimen is higher or lower than normal, indicating the tissue is dehydrated or over hydrated respectively.

The indicator unit provides the information in terms of hydration level computed from the measured osmolality, instead of, or in addition, to the osmolality value.

The indicator unit consists of a wrist-watch device, which in addition of conventional watch functions, provides the user with osmolality/hydration level information.

The measurement device transmits the osmolality/hydration information, to a separate display unit, such as a cellular phone. The cellular phone transmits the osmolality/hydration information obtained to a preset medical service

The processor is adapted to store the intensity of the RF signals, at the plurality of frequencies, reflected from the tissue, under standard normal osmolality conditions, for use as the at least one reference value.

The processor is preset with the at least one reference values, in accordance with known normal body fluid osmolality.

The transmitter and receiver have separate antennae serving the transmission and reception functions of the system.

A single antenna is used only for transmission, wherein the system includes a transmitter equipped with a standing wave ratio (SWR) metering function, wherein the SWR values at the various frequencies used, are fed to the aforementioned processor, instead of the aforementioned reflection intensities.

The at least one reference value includes at least one reflection ratio or SWR ratio.

The transmitter is adapted to measure the standing wave ratio (SWR) induced in the transmission circuit by the reflection of the signal from the tissue.

A method for non-invasive measurement of the state of hydration in a living specimen, the method includes transmitting RF signals, at a plurality of frequencies, towards the tissue, receiving the RF signals reflected from the tissue and measuring the intensities thereof, feeding the reflection intensities thus measured to the processor, which uses the intensities to compute the frequency dependence of the reflection intensities, comparing the frequency dependence of the reflection intensities thus obtained, with at least one reference set of reflection signal frequency dependence, and deriving from the comparison the hydration state of the tissue of interest. The method may provide an output signal indicative of a hydration state of the living specimen.

In accordance with non-limiting embodiments of the invention:
The reference set of reflection signal frequency dependence is obtained by transmitting RF signals towards the same tissue of the same user when subjected to standard hydration conditions.

The output signal is an audible, visual or palpating signal.

The RF signals are transmitted towards the tissue at the same frequencies as frequencies of the reference signals.

The RF signals are transmitted towards the tissue includes transmission towards the wrist.

The RF signals are transmitted towards the tissue, such as towards a member of a living body.

The measurement consists of transmission of the RF signals towards the tissue and measuring the standing wave ratio (SWR) of the transmitted RF signal.

The output is used to operate automatically an osmolality correcting device (e.g. —a water pump) to bring the osmolality in the organ under examination back to the normal range.

### BRIEF DESCRIPTION OF THE DRAWINGS

The present invention will be understood and appreciated more fully from the following detailed description taken in conjunction with the drawings in which:

FIG. 1 is a simplified block diagram of the system according to the present invention for measuring the osmolality of fluids or hydration level of a living body.

### DETAILED DESCRIPTION OF EMBODIMENTS

The invention will now be described in connection with certain preferred embodiments with reference to the following illustrative figure, so that it may be more fully understood.

With specific reference now to the figure in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawing making apparent to those skilled in the art how the several forms of the invention may be embodied in practice.

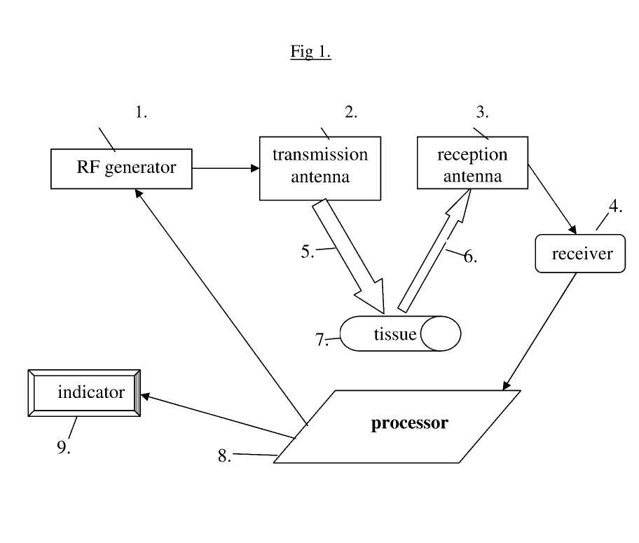

Seen in FIG. 1 is a system which includes an RF generator 1 connected to a transmitting antenna 2 for transmitting the generated RF radiation waves 5 towards the examined tissue 7, a reception antenna 3 for receiving the waves 6 reflected from the examined tissue, a processor 8 for processing the signal picked up by the reception antenna, and an indicator unit 9 for displaying or otherwise providing an output signal, e.g., an alarm, indicative of the degree of osmolality/hydration state of the body. Said processor serves as a controller of the RF generator 1, to initiate the generation of a measurement process.

In the method, the processor 8 controls the RF generator 1, and initiates transmission of RF waves 5 of at least two frequencies in sequence from the transmission antenna 2, towards the examined tissue member 7. The reflected waves 6 are picked up by the reception antenna 3, which is connected to the receiver 4, which analyzes the intensity of the reflected waves. from here, the intensities thus measured are passed to the processor 8 to derive the osmolality by comparing the ratio of intensities of the reflected wave intensities at the various frequencies, and comparing these ratios to the same ratios obtained from standard tissue with normal osmolality, or alternatively from the same tissue member of the same person, when known to be in euhydrated state.

The osmolality/hydration level thus computed is passed over to the display/indicator unit 9, which in case of deviation from normal values emits warning signals.

According to a preferred embodiment of the present invention, the frequencies used are:—“high”=2.45 GHz and “low”=40.68 MHz,

It should be noted that using more than two frequencies improves accuracy and reliability, and the above-described embodiment has been presented for reasons of simplicity only.

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

What is claimed is:

1. A system for non-invasive in-vivo, measurement of the osmolality of biological tissue fluid in a living specimen, said system comprising:
an RF generator/transmitter for emitting RF radiation signals at a plurality of frequencies and for transmitting said radiation signals towards a tissue of a living specimen;
a receiver adapted to receive RF radiation signals reflected from the tissue of the living specimen at the plurality of frequencies; and
a processor unit operative to receive signals from said receiver and to compare intensity of the RF radiation signals at the plurality of frequencies reflected from said tissue of the living specimen, with that of at least one reference value obtained from tissue with a known normal osmolality, and based on that, compute osmolality of said tissue of the living specimen.

2. The system according to claim 1, further comprising an indicator unit for providing an output signal representative of the osmolality of the fluid of said tissue, and issuing a warning signal if the osmolality value is significantly out of normal range.

3. The system according to claim 1, wherein said RF generator/transmitter emits RF radiation at frequencies of between 10 kHz and 300 GHz.

4. The system according to claim 1, further comprising an attachment for attaching said system to a part of a user's body.

5. The system according to claim 1, wherein the plurality of frequencies include at least one frequency at which RF energy impinging on the tissue induces mainly ionic motion of ions dissolved in water in the tissue.

6. The system according to claim 1, wherein the plurality of frequencies include at least one frequency at which RF energy causes mainly rotational excitation of tissue water molecules.

7. The system according to claim 2, wherein the indicator unit comprises a wrist-watch device, which in addition to conventional watch functions, provides osmolality/hydration level information.

8. The system according to claim 1, further comprising a unit that transmits osmolality/hydration information to a separate display unit.

9. The system according to claim 1, wherein the processor is adapted to store the intensity of the RF signals, at the plurality of frequencies, reflected from said tissue, under standard normal osmolality conditions, for use as the at least one reference value.

10. The system according to claim 1, wherein said RF generator/transmitter comprises a standing wave ratio (SWR) metering function.

11. The system according to claim 10, wherein said transmitter is adapted to measure the standing wave ratio (SWR) induced in a transmission circuit by reflection of the signal from said tissue.

12. A method for non-invasive measurement of the state of hydration in a living specimen, the method comprising:
transmitting RF signals, at a plurality of frequencies, towards a tissue of a living specimen;
receiving RF signals reflected from said tissue and measuring the reflection intensities thereof;
using said reflection intensities to compute a frequency dependence of the reflection intensities;
comparing the frequency dependence of the reflection intensities thus obtained, with at least one reference set of reflection signal frequency dependence; and
deriving from said comparison the hydration state of the tissue of the living specimen.

13. The method according to claim 12, providing an output signal indicative of a hydration state of the living specimen.

14. The method according to claim 12, wherein said reference set of reflection signal frequency dependence is obtained by transmitting RF signals towards the same tissue of the same user when subjected to standard hydration conditions.

15. The method according to claim 13, wherein said output signal is an audible, visual or palpating signal.

16. The method according to claim 12, wherein said RF signals are transmitted towards the tissue at the same frequencies as frequencies of said reference signals.

17. The method according to claim 12, comprising transmitting the RF signals towards a wrist of a user.
